(19) United States
(12) Patent Application Publication
(10) Pub. No.: US 2025/0109095 A1
(43) Pub. Date: Apr. 3, 2025

(54) METHOD FOR PREPARING OXIME DERIVATIVE

(71) Applicant: SEOUL NATIONAL UNIVERSITY R&DB FOUNDATION, Seoul (KR)

(72) Inventors: Yun Ho LEE, Seoul (KR); Jin Seong GWAK, Seoul (KR); Jong Hoon CHOI, Daejeon (KR); Sudakar PADMANABAN, Seoul (KR)

(73) Assignee: SEOUL NATIONAL UNIVERSITY R&DB FOUNDATION, Seoul (KR)

(21) Appl. No.: 18/714,850

(22) PCT Filed: Nov. 24, 2022
(86) PCT No.: PCT/KR2022/018688

§ 371 Date: May 30, 2024

(30) Foreign Application Priority Data
Nov. 30, 2021 (KR) 10-2021-0169457

(51) Int. Cl. C07C 249/06; B01J 31/18; B01J 31/24

(52) U.S. Cl. CPC C07C 249/06; B01J 31/1825; B01J 31/2409; B01J 2531/847; C07C 2601/14; C07C 2603/18

(57) ABSTRACT

The present disclosure relates to a method for more readily preparing an oxime derivative under mild conditions by using a nitrogen oxide as a NO supply source.

### TECHNICAL FIELD

The present disclosure claims priority to and the benefits of Korean Patent Application No. 10-2021-0169457, filed with the Korean Intellectual Property Office on Nov. 30, 2021, the entire contents of which are incorporated herein by reference. The present disclosure relates to a method for preparing an oxime derivative.

### BACKGROUND ART

Nitrogen oxides (NOₓ) emitted from human activities are major pollutants and cause serious environmental problems. Emissions in the automotive industry cause NO₂ and NO pollution, however, widespread use of nitrate (NO₃⁻)-based fertilizers in the agricultural sector increases dystrophication, causing dead spots having serious impacts on the ecosystem. Significant efforts are needed on NOₓ conversion in order to find appropriate solutions for balancing the impact of NOₓ and the global nitrogen cycle. Nitrate, one of the most common nitrogen oxyanions, is inert to reduction due to weak complexation and poor nucleophilicity. For activation, harsh reaction conditions such as low pH, high temperature and photolysis are required. NO₂ and NO are main toxic substances of exhaust gases and are difficult to reduce since they are generally encountered in highly oxidized environments such as an engine combustion chamber. Selective reduction using ammonia with expensive precious metals such as Pt and Rh as a catalyst normally takes place, however, inefficiency and energy costs of this technology are still problems. Nitrous oxide (N₂O) is also one of greenhouse gases, and has a heat capturing ability 300 times higher than carbon dioxide (CO₂). Unfortunately, its conversion is hindered by highly inert properties. This may be overcome through bonding and activation by metal ions, however, intrinsic weak c-donating and π-accepting abilities of N₂O strengthen a kinetic barrier. Conversion of nitrate into dinitrogen (N₂) in nature occurs through a sequential deoxygenation pathway (NO₃⁻->NO₂⁻/NO->N₂O->N₂) known as a retrograde process of the global nitrogen cycle or a denitrification process. Each enzymatic reaction occurs effectively under biological conditions, however, unfortunately, the process requires four different metalloenzymes of nitrate reductase, nitrite reductase, nitric oxide reductase and nitrous oxide reductase, leading to a problem of making economical industrial solutions difficult.

### DISCLOSURE

### Technical Problem

The present disclosure is directed to providing a method for preparing an oxime derivative having excellent yield and oxime selectivity by using a nitrogen oxide as a NO supply source under mild conditions.

However, objects to be addressed by the present disclosure are not limited to the object mentioned above, and other objects not mentioned will be clearly appreciated by those skilled in the art from the following description.

### Technical Solution

One embodiment of the present disclosure provides a method for preparing oxime and its derivatives, the method including preparing a second compound represented by the following Chemical Formula 2 from a first compound represented by the following Chemical Formula 1, wherein the preparing of a second compound uses a Ni-based catalyst represented by the following Chemical Formula 3:

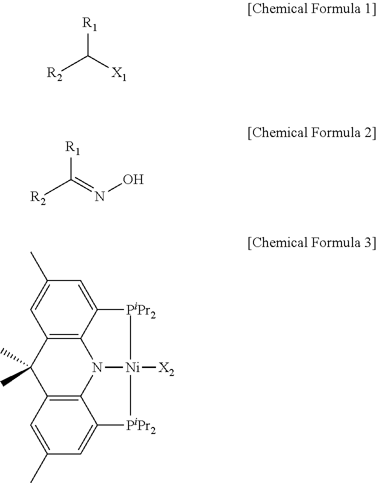

in Chemical Formula 1 to Chemical Formula 3,
R₁ and R₂ are each independently hydrogen; an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms, or
R₁ and R₂ are linked to each other to form an alicyclic ring having 5 to 12 carbon atoms; or an alicyclic ring having 5 to 12 carbon atoms to which an aromatic ring having 6 to 30 carbon atoms bonds, and
X₁ and X₂ are each independently F; Cl; Br; or I,
in Chemical Formula 3, ⁱPr represents an iso-propyl group or related substituent, and
in the substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ is an alkyl group having 1 to 3 carbon atoms.

### Advantageous Effects

A method for preparing an oxime derivative according to one embodiment of the present disclosure can prepare an oxime derivative more readily by using a nitrogen oxide as a NO supply source under mild conditions.

A method for preparing an oxime derivative according to one embodiment of the present disclosure can have excellent yield and oxime selectivity.

Effects of the present disclosure are not limited to the above-described effects, and effects not mentioned will be clearly appreciated by those skilled in the art from the present specification and accompanying drawings.

### BRIEF DESCRIPTION OF DRAWINGS

FIG. 1 shows images of crystal structures of Compound 3-1 to Compound 3-3 according to the present disclosure. Specifically, FIG. 1 (a) is an image showing a crystal structure of Compound 3-1, FIG. 1 (b) is an image showing a crystal structure of Compound 3-2 and FIG. 1 (c) is an image showing a crystal structure of Compound 3-3 (displacement ellipsoids are set at 50% probability, and hydrogen atoms are omitted for clarity).

FIG. 2 shows structural data for nickel-nitrosyl moieties of Compound 3-3 and Compound 3-3′ according to the present disclosure and Ni K-edge X-ray absorption spectrum images of Compound 3-3 and Compound 3-3′ with respect to {(^{acri}PNP)Ni^{II}(CO)}{BF₄}, (^{acri}PNP)Ni^{I}(CO) and {(^{acri}PNP)Ni⁰(CO)}{Na}.

FIG. 3 schematically shows two different reaction paths of a catalytic reaction according to the present disclosure.

FIG. 4 to FIG. 28 are ¹H NMR analysis images for products according to Example 1 to Example 5, Example 8 to Example 14, Example 16 to Example 25 and Comparative Example 1 to Comparative Example 3.

### MODE FOR INVENTION

Throughout the present specification, a description of a certain part “including” certain constituents means that it may further include other constituents, and does not exclude other constituents unless particularly stated on the contrary.

Hereinafter, the present disclosure will be described in more detail.

One embodiment of the present disclosure provides a method for preparing an oxime derivative, the method including preparing a second compound represented by the following Chemical Formula 2 from a first compound represented by the following Chemical Formula 1, wherein the preparing of a second compound uses a Ni-based catalyst represented by the following Chemical Formula 3:

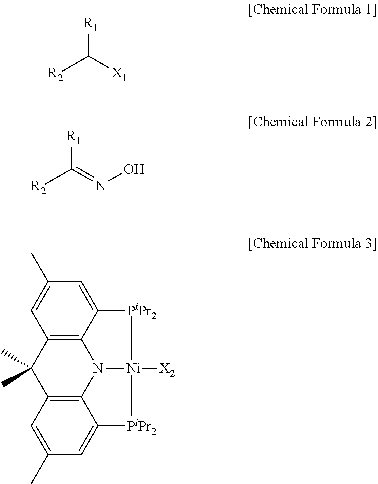

in Chemical Formula 1 to Chemical Formula 3, R₁ and R₂ are each independently hydrogen; an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms, or R₁ and R₂ are linked to each other to form an alicyclic ring having 5 to 12 carbon atoms; or an alicyclic ring having 5 to 12 carbon atoms to which an aromatic ring having 6 to 30 carbon atoms bonds, and X. and X, are each independently F; Cl; Br; or I, ⁱPr in Chemical Formula 3 represents an iso-propyl group or related substituent, and, in the substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ is an alkyl group having 1 to 0.3 carbon atoms.

The method for preparing an oxime derivative according to one embodiment of the present disclosure uses nitrogen oxide (NOₓ) as a nitric oxide (NO) supply source under mild conditions, and through a catalytic reaction of transferring a nitroso group to the first compound represented by Chemical Formula 1 using the Ni-based catalyst, oxime derivatives may be more readily prepared. In addition, the method for preparing an oxime derivative according to one embodiment of the present disclosure may have excellent yield and oxime selectivity.

According to one embodiment of the present disclosure, the first compound represented by Chemical Formula 1 may include R₁ and R₂ that are each independently hydrogen; an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms. Specifically, R₁ and R₂ may be substituents that are the same as or different from each other, and R₁ may be hydrogen and R₂ may be an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms. More specifically, when R₂ is a substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ, may be an alkyl group having 1 to 3 carbon atoms. Herein, Rₐ may be a methyl group. When R₁ and R₂ are substituents of the above-described types, NO is readily transferred by the Ni-based catalyst, and therefore, an oxime derivative may be readily prepared.

According to one embodiment of the present disclosure, R₁ and R₂ may be linked to each other to form an alicyclic ring having 5 to 12 carbon atoms; or an alicyclic ring having 5 to 12 carbon atoms to which an aromatic ring having 6 to 30 carbon atoms bonds. When R₁ and R₂ are linked to form a ring structure of the above-described type, steric hindrance around the benzyl position increases, resulting in excellent selectivity of catalytic NO transfer using the Ni-based catalyst of the present disclosure, and accordingly, yield and turnover number (TON, number of moles of oxime formed per mole of Ni catalyst) of an oxime derivative may increase.

According to one embodiment of the present disclosure, X₁ may be F; Cl; Br; or I. Specifically, X₁ may be Cl; or Br. More specifically, X₁ may be Br. When X₁ is a halogen atom of the above-described type, NO is readily transferred to the compound represented by Chemical Formula 1 by the Ni-based catalyst, and therefore, oxime derivatives may be readily prepared.

According to one embodiment of the present disclosure, in Chemical Formula 1, X₁ is Br, R₁ and R₂ are each independently hydrogen; or an unsubstituted or substituted aryl group having 6 to 30 carbon atoms, and in the substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and T; or —CO₂Rₐ, and Rₐ may be an alkyl group having 1 to 3 carbon atoms. Herein, Rₐ may be a methyl group.

In addition, in Chemical Formula 1, at least one of R₁ and R₂ is

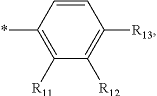

and R₁₁ to R₁₃ are each independently hydrogen; F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ may be an alkyl group having 1 to 3 carbon atoms. “*” represents a bonding position. Specifically, R₁₁ and R₁₂ are each independently F; Cl; Br; I; an alkyl group having 1 to 3 carbon atoms unsubstituted or substituted with at least one of F, Cl, Br and I; or an alkoxy group having 1 to 3 carbon atoms unsubstituted or substituted with at least one of F, Cl, Br and I, and R₁₃ may be hydrogen. When R₁₁ to R₁₃ are the above-described types, that is, do not have substituents at a para position, yield and turnover number of an oxime derivative may increase. Specifically, R₁₁ is hydrogen, and R₁₂ and R₁₃ are each independently F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ may be an alkyl group having 1 to 3 carbon atoms. When R₁₁ to R₁₃ are the above-described types, that is, do not have substituents at an ortho position, NO is readily transferred to the compound represented by Chemical Formula 1 by the Ni-based catalyst, and therefore, an oxime derivative may be readily prepared.

In addition, in Chemical Formula 1, X₁ is Br, and R₁ and R₂ may be linked to each other to form an alicyclic ring having 5 to 12 carbon atoms; or an alicyclic ring having 5 to 12 carbon atoms to which an aromatic ring having 6 to 30 carbon atoms bonds. When X₁, R₁ and R₂ are the above-described types, selectivity of catalytic NO transfer using the Ni-based catalyst may be excellent, and accordingly, yield and turnover number of an oxime derivative may increase.

According to one embodiment of the present disclosure, in Chemical Formula 1, R₁ and R₂ may be linked to each other to form

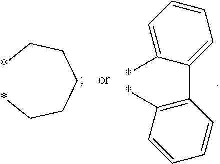 [SMILES: *CCCCC* ; *c1ccccc1-c1ccccc1*]

“*” represents a bonding position. When R₁ and R₂ are linked to each other to form a ring structure of the above-described type, steric hindrance around the benzyl position increases, resulting in excellent selectivity of catalytic NO transfer using the Ni-based catalyst of the present disclosure, and accordingly, yield and turnover number of an oxime derivative may increase.

According to one embodiment of the present disclosure, the first compound may be one selected from among compounds represented by the following Chemical Formula 1-1 to Chemical Formula 1-10:

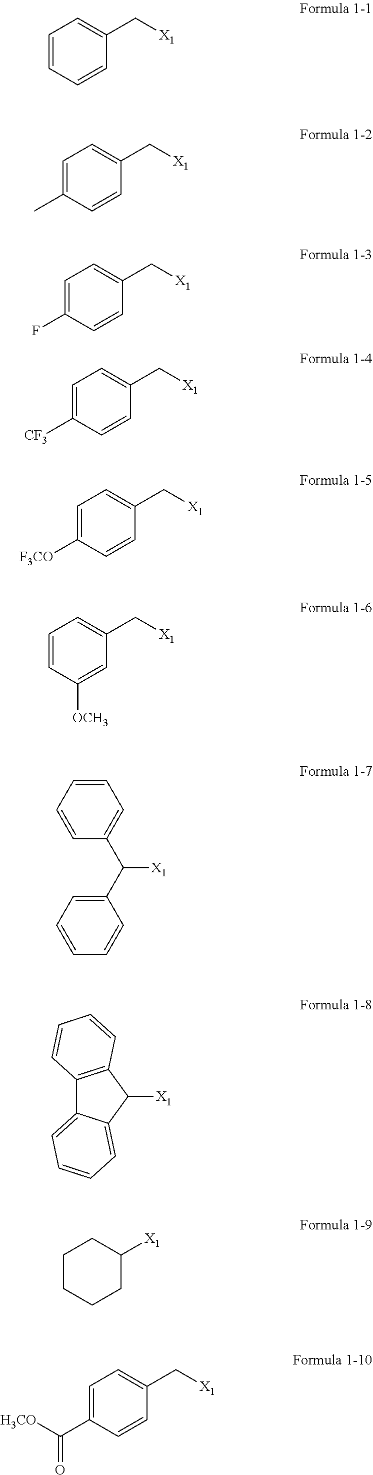

in Chemical Formula 1-1 to Chemical Formula 1-10, X₁ is F; Cl; Br; or I.

When the first compound is a compound of the above-described type, NO is readily transferred to the compound represented by Chemical Formula 1 by the Ni-based catalyst, and therefore, an oxime derivative may be readily prepared.

According to one embodiment of the present disclosure, in Chemical Formula 1-1 to Chemical Formula 1-10, X₁ may be Cl; or Br. Specifically, X₁ may be Br. In other words, the first compound may be one selected from among the following Compound 1-1 to Compound 1-10.

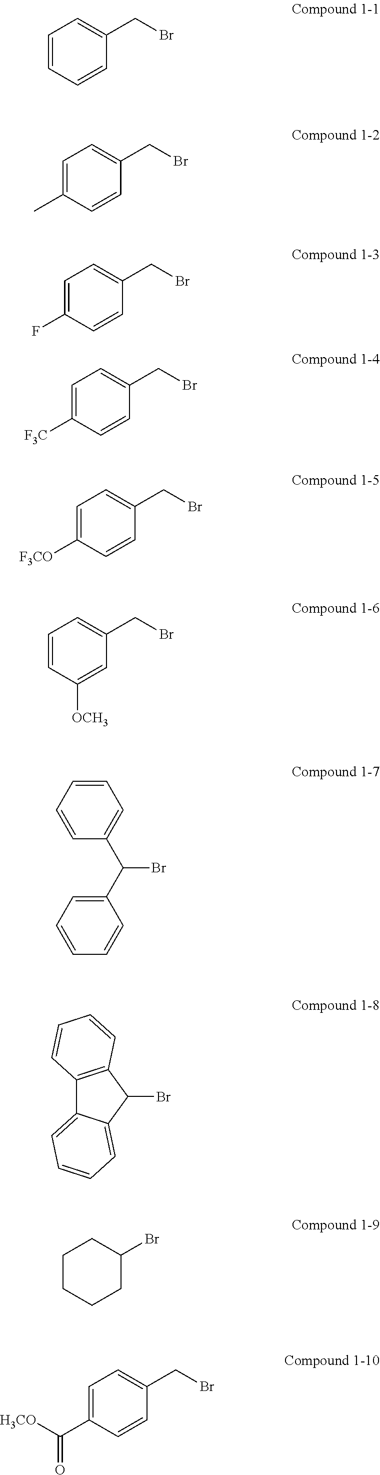

When the first compound in which X₁ is Cl or Br is used, the halogen element is more readily removed, facilitating NO transfer by the Ni-based catalyst, and as a result, an oxime derivative may be more readily selectively prepared.

According to one embodiment of the present disclosure, the preparing of a second compound may be forming the second compound from the first compound using a mixture solution including the Ni-based catalyst, nitrogen oxide (NOₓ) and a solvent.

According to one embodiment of the present disclosure, the Ni-based catalyst may be a compound represented by the following Chemical Formula 3.

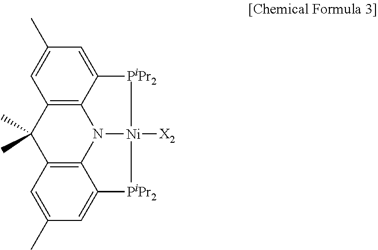

In Chemical Formula 3, ⁱPr represents an iso-propyl group, and X₂ may be F; Cl; Br; or I. Specifically, X₂ may be Cl. In other words, the Ni-based catalyst may be the following Compound 3-5.

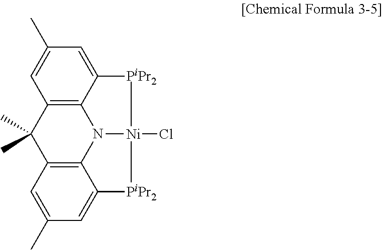

When X₂ is a halogen element of the above-described type, efficiency of the catalytic cycle of the compound represented by Chemical Formula 3 shown in Reaction Formula 1 to describe later may be more superior, and NO transfer and catalyst regeneration of the Ni-based catalyst may more readily occur.

In addition, Compound 3-5 may form the following Compound 3-3 according to the catalytic cycle shown in the following Reaction Formula 1.

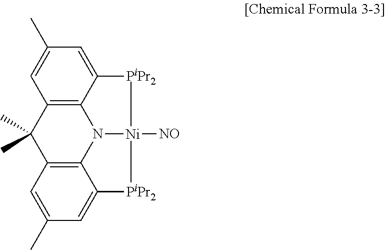

When the Ni-based catalyst is a compound of the above-described type, NO transfer efficiency and oxime selectivity using the Ni-based catalyst may be more superior, and efficiency of the catalytic cycle and catalytic reaction shown in the following Reaction Formula 1 may be more superior.

According to one embodiment of the present disclosure, the Ni-based catalyst may form Compound 3-1 to Compound 3-4 according to a catalytic cycle of the following Reaction Formula 1 in a mixture solution including a nitrogen oxide and a solvent, and Compound 3-3 may transfer a nitroso group to the first compound represented by Chemical Formula 1 according to the following Reaction Formula 2 to induce a catalytic reaction of preparing the second compound represented by Chemical Formula 2.

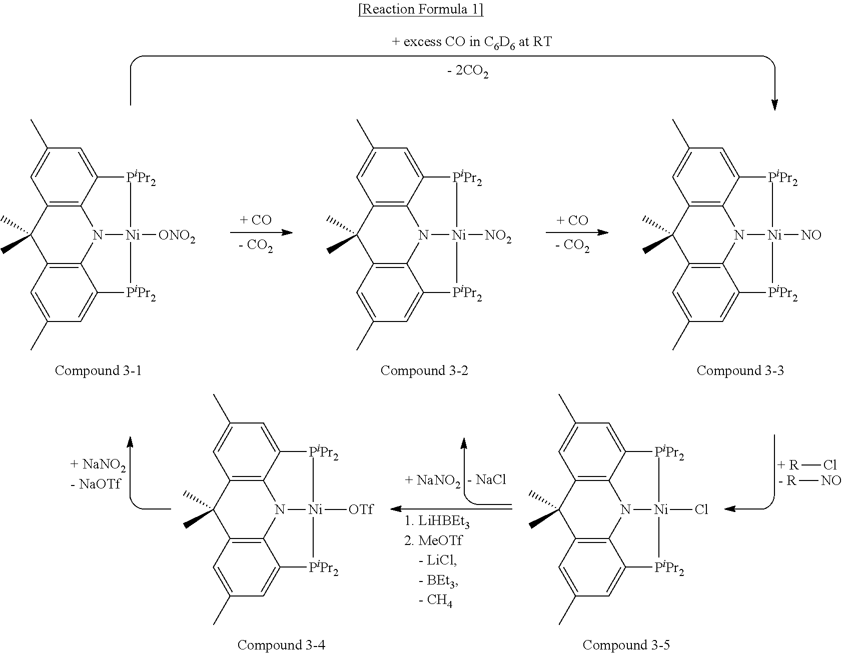

In Reaction Formula 1, Compound 3-1 is (^{acri}PNP)Ni(NO₃), Compound 3-2 is (^{acri}PNP)Ni(NO₂), Compound 3-3 is (^{acri}PNP)Ni(NO), Compound 3-4 is (^{acri}PNP)Ni(OTf) and Compound 3-5 is (^{acri}PNP)Ni(Cl), and herein, ^{acri}PNP represents a pincer-type ligand that is 4,5-bis(diisopropylphosphino)-2,7,9,9-tetramethyl-9H-acridin-10-ide). The structure of the ligand may have some changes in the structure such as substituents within a range of having properties of the Ni catalyst for NO transferring of the present disclosure.

According to one embodiment of the present disclosure, the nitrogen oxide may have a form of NO₂, that is, NO, NO₂ and NO₃. Specifically, the nitrogen oxide may have a form of NO₂, and more specifically, the nitrogen oxide may have a form of nitrogen oxyanion. More specifically, the nitrogen oxide may have a form of NaNO₂. When the nitrogen oxide is the above-described type, nitrogen oxides (NOₓ), one of major pollutants emitted from human activities, may be utilized in various forms for the catalytic reaction, and therefore, more economical industrial solutions may be provided. In addition, through the catalytic reaction, highly reactive nitrogen oxides may be stabilized, and utilized for further conversion into value-added products.

According to one embodiment of the present disclosure, the first compound and the nitrogen oxide may have a molar ratio of greater than or equal to 1:2 and less than 1:4. Specifically, the first compound and the nitrogen oxide may have a molar ratio of greater than or equal to 1:2 and less than or equal to 1:3.5, greater than or equal to 1:2 and less than or equal to 1:3, greater than or equal to 1:2.5 and less than or equal to 1:3.5 or greater than or equal to 1:1.25 and less than or equal to 1:3. When the molar ratio between the first compound and the nitrogen oxide satisfies the above-described range, the halogen element included in the Ni-based catalyst may be readily converted into a nitroso group, and side reactions may be reduced while readily transferring NO to the compound represented by Chemical Formula 1 by the Ni-based catalyst. Accordingly, oxime selectivity and turnover number may be enhanced during the oxime derivative preparation process.

According to one embodiment of the present disclosure, the solvent may include at least one of acetone, tetrahydrofuran, propylene carbonate, 1,4-dioxane, toluene and acetonitrile. Specifically, the solvent may be acetone or tetrahydrofuran. When the solvent of above-described type is used, side reactions may be reduced while readily transferring NO to the compound represented by Chemical Formula 1 by the Ni-based catalyst. Accordingly, oxime selectivity and turnover number may be enhanced during the oxime derivative preparation process.

According to one embodiment of the present disclosure, the mixture solution may further include a crown ether. Specifically, the crown ether may be 18-crown-6, dicyclohexyl-18-crown-6, dibenzo-18-crown-6, 15-crown-5, 12-crown-4, benzo-15-crown-5, benzo-18-crown-6, 4′-aminobenzo-18-crown-6 or 4′-nitrobenzo-15-crown-5. More specifically, the crown ether may be 15-crown-5. When the crown ether of above-described type is included, the compound represented by Chemical Formula 1 and the Ni-based catalyst may readily react in selecting a solvent for reducing the side reactions, and accordingly, selectivity and turnover number of an oxime derivative may be further enhanced.

According to one embodiment of the present disclosure, the first compound and the crown ether may have a molar ratio of greater than or equal to 1:12.5 and less than or equal to 1:50. Specifically, the first compound and the crown ether may have a molar ratio of greater than or equal to 1:12.5 and less than or equal to 1:40, greater than or equal to 1:12.5 and less than or equal to 1:30, greater than or equal to 1:12.5 and less than or equal to 1:20, greater than or equal to 1:20 and less than or equal to 1:50, greater than or equal to 1:30 and less than or equal to 1:50 or greater than or equal to 1:20 and less than or equal to 1:40. When the molar ratio between the first compound and the crown ether satisfies the above-described range, the compound represented by Chemical Formula 1 and the Ni-based catalyst may more readily react.

According to one embodiment of the present disclosure, the preparing of a second compound may be performed for a period of 24 hours or more and 48 hours or less at a temperature of 25° C. or higher and 80° C. or lower under the CO atmosphere. Specifically, the preparing of a second compound may be performed at a temperature of 25° C. or higher and 70° C. or Lower, 25° C. or higher and 60° C. or lower, 25° C. or higher and 50° C. or lower, 25° C. or higher and 60° C. or lower, 40° C. or higher and 80° C. or lower, 50° C. or higher and 80° C. or lower, 40° C. or higher and 70° C. or lower or 50° C. or higher and 60° C. or lower. Specifically, the preparing of a second compound may be performed for a period of 30 hours or more and 48 hours or less, 36 hours or more and 48 hours or less, 42 hours or more and 48 hours or less, 24 hours or more and 42 hours or less, 24 hours or more and 36 hours or less or 24 hours or more and 30 hours or less. In addition, the reaction may be performed under mild conditions of lower temperatures by increasing the reaction time, or the reaction may be performed at higher temperatures by decreasing the reaction time. When the reaction condition satisfies the above-described range, the NO transfer catalytic reaction using the Ni-based catalyst may occur under milder conditions, and oxime selectivity and turnover number may be more superior.

According to one embodiment of the present disclosure, the second compound represented by Chemical Formula 2 may be an oxime derivative in which, in Chemical Formula 2, R₁, R₂, and R₁₁ to R₁₃ have the same definitions as in Chemical Formula 1. Specifically, the second compound represented by Chemical Formula 2 may include R₁ and R₂ that are each independently hydrogen; an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms. Specifically, R₁ and R₂ may be substituents that are the same as or different from each other, and R₁ may be hydrogen and R₂ may be an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms. More specifically, when R₂ is a substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ may be an alkyl group having 1 to 3 carbon atoms. When R₁ and R₂ are substituents of the above-described types, NO transfer by the Ni-based catalyst and tautomerization to an oxime derivative are facilitated, and selectivity of the oxime derivative may be more superior.

According to one embodiment of the present disclosure, R₁ and R₂ may be linked to each other to form an alicyclic ring having 5 to 12 carbon atoms; or an alicyclic ring having 5 to 12 carbon atoms to which an aromatic ring having 6 to 30 carbon atoms bonds. When R₁ and R₂ are linked to each other to form a ring structure of the above-described type, steric hindrance around the benzyl position increases, facilitating catalytic NO transfer using the Ni-based catalyst of the present disclosure and tautomerization to an oxime derivative, and accordingly, yield and turnover number of the oxime derivative may increase.

According to one embodiment of the present disclosure, in Chemical Formula 2, R₁ and R₂ are each independently hydrogen; or an unsubstituted or substituted aryl group having 6 to 30 carbon atoms, and in the substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ may be an alkyl group having 1 to 3 carbon atoms.

In addition, in Chemical Formula 2, at least one of R₁ and R₂ is

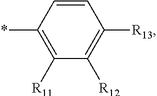

and R₁₁ to R₁₃ are each independently hydrogen; F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ may be an alkyl group having 1 to 3 carbon atoms. “*” represents a bonding position. Specifically, R₁₁ and R₁₂ are each independently F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, Rₐ may be an alkyl group having 1 to 3 carbon atoms, and R₁₃ may be hydrogen. When R₁₁ to R₁₃ are the above-described types, that is, do not have substituents at a para position, yield and turnover number of an oxime derivative may increase. Specifically, R₁₁ is hydrogen, R₁₂ and R₁₃ are each independently F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ may be an alkyl group having 1 to 3 carbon atoms. When R₁₁ to R₁₃ are the above-described types, that is, do not have substituents at an ortho position, NO transfer by the Ni-based catalyst and tautomerization to an oxime derivative are facilitated, and therefore, an oxime derivative may be more readily prepared.

In addition, in Chemical Formula 2, R₁ and R₂ may be linked to each other to form an alicyclic ring having 5 to 12 carbon atoms; or an alicyclic ring having 5 to 12 carbon atoms to which an aromatic ring having 6 to 30 carbon atoms bonds. When R₁ and R₂ are the above-described types, selectivity of catalytic NO transfer using the Ni-based catalyst may be excellent, and as a result, yield and turnover number an oxime derivative may increase.

According to one embodiment of the present disclosure, in Chemical Formula 2, R₁ and R₂ may be linked to each other to form

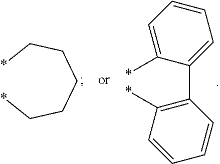 [SMILES: *CCCCC* ; *c1ccccc1-c1ccccc1*]

“*” represents a bonding position. When R₁ and R₂ are linked to each other to form a ring structure of the above-described type, steric hindrance around the benzyl position increases, facilitating the catalytic NO transfer reaction using the Ni-based catalyst of the present disclosure and tautomerization to an oxime derivative, and accordingly, yield and turnover number of the oxime derivative may Increase. In addition, the reaction for preparing the oxime derivative may be performed under milder conditions.

According to one embodiment of the present disclosure, the second compound may be one selected from among the following Compound 2-1 to Compound 2-10.

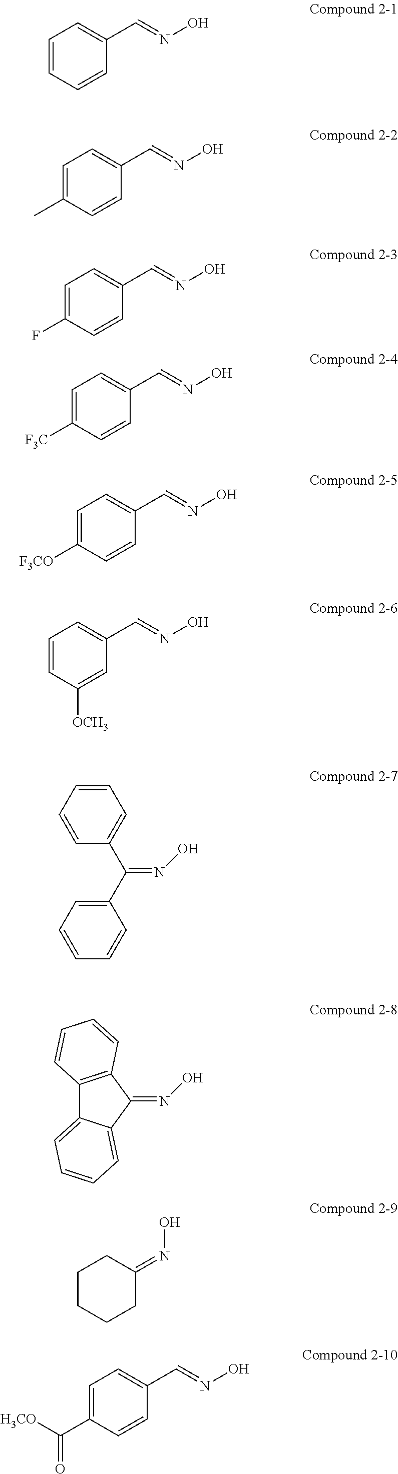

When the second compound is the above-described type, NO transfer by the Ni-based catalyst may be facilitated, and tautomerization may be facilitated, and as a result, an oxime derivative may be more readily selectively prepared. Accordingly, stabilization of highly reactive nitrogen oxides through the catalytic reaction and further conversion into value-added products may be facilitated, and more economical industrial solutions may be provided.

Hereinafter, the present disclosure will be described in detail with reference to examples in order to specifically describe the present disclosure. However, examples according to the present disclosure may be modified to various different forms, and the scope of the present disclosure is not construed as being limited to the examples described below. Examples of the present specification are provided in order to more fully describe the present disclosure to those having average knowledge in the art.

### Preparation Example: Preparation of ^{acri}PNP Ligand (^{acri}PNP-″4,5-bis(diisopropylphosphino)-2,7,9,9-tetramethyl-9H-acridin-10-ide)-based Ni(NO) Complex/Preparation of Compound 3-3

(^{acri}PNP)Ni(NO₃) (Compound 3-1), (^{acri}PNP)Ni(NO₂) (Compound 3-2) and (^{acri}PNP)Ni(NO) (Compound 3-3) were prepared based on a synthesis protocol reported as follows (Chem. Sci., 2019, 10, 4767-4774). Compound 3-1 and Compound 3-2 were synthesized through an anion metathesis reaction of (^{acri}PNP)Ni(X) (Compound 3-4; X═OTf, trifluoromethanesulfonate, or Compound 3-5; X═Cl) using sodium nitrate and sodium nitrite, respectively. Two diamagnetic products, which exhibit a singlet at about 45 ppm in a ³¹P NMR spectrum of C₆D₆, were separated as powders in a yield of about 90%. It was identified that, whereas IR spectrum of Compound 3-1 showed NO stretching vibrations of the nitrate moiety at 1,280 cm⁻¹ and 1,000 cm⁻¹, IR data of Compound 3-2 showed, similar to other nickel(II) nitro complexes, asymmetric stretching vibration of the nitrite moiety at 1,373 cm⁻¹ and symmetric stretching vibration at 1,325 cm⁻¹.

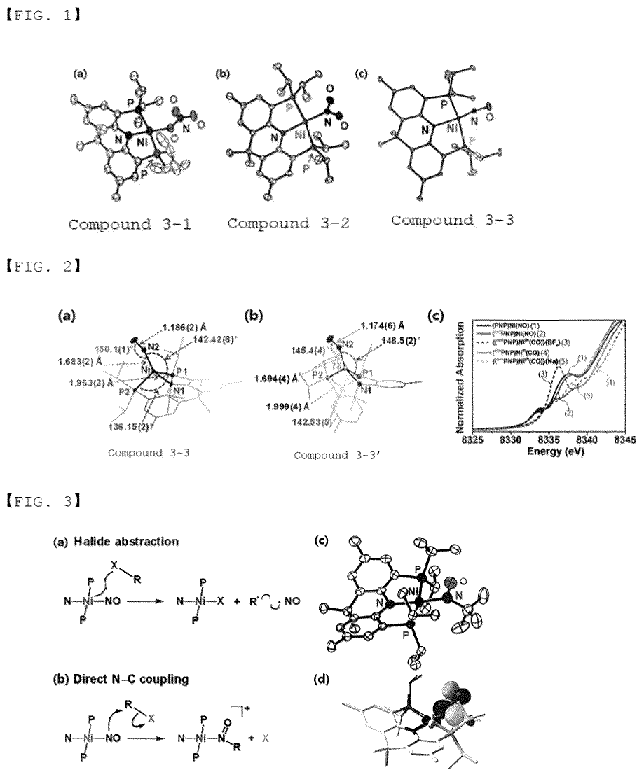

FIG. 1 shows images of crystal structures of Compound 3-1 to Compound 3-3 according to the present disclosure. Specifically, FIG. 1 (a) is an image showing a crystal structure of Compound 3-1, FIG. 1 (b) is an image showing a crystal structure of Compound 3-2 and FIG. 1 (c) is an image showing a crystal structure of Compound 3-3 (displacement ellipsoids are set at 50% probability, and hydrogen atoms are omitted for clarity).

Referring to FIG. 1, it was identified that one of the oxygen atoms in the NO₃⁻ moiety was coordinated to a square planar nickel(II) center (τ₄₌0.09) having a bond distance of 1.918(3) Å. The structure of Compound 3-2 represents a nickel(II) nitro complex having a Ni—N bond of 1.847(1) Å. It was identified that the crystal structure data were similar to those of (PNP)Ni(NO₃) (Compound 3-1′) and (PNP)Ni(NO₂) (Compound 3-2′), similar nickel nitrate and nitro complexes previously reported, respectively.

For Compound 3-1 and Compound 3-2, deoxygenation of Ni-bonding nitrogen oxyanion included in the Ni—NO by CO (g) was identified. According to study results using (PNP)Ni scaffolds, conversion of Ni—NO₃ species to corresponding Ni—NO species includes formation of stable and isolable nickel nitrite intermediate species. As a result of monitoring the deoxygenation by CO (g) using ³¹P NMR spectroscopy as above, it was identified that the singlet of Compound 3-2 appeared at 47.9 ppm, whereas the singlet of Compound 3-1 decreased at 42.3 ppm. Meanwhile, appearance of one signal at 56.7 ppm was identified, and it may be seen that this corresponds to (^{acri}PNP)Ni(NO) (Compound 3-3), a nickel nitrosyl compound.

Compound 3-3 was synthetically prepared by adding CO (g) to a brown solution obtained by adding Compound 3-2 to benzene at room temperature, and separated as a brown powder in a yield of 98%. Through the fact that Compound 3-2 was also cleanly produced when 1 equivalent of CO(g) was added to Compound 3-1 and Compound 3-2 was able to be directly converted into Compound 3-3 using CO (g) as described above, it may be seen that a nickel nitrite complex was formed as an intermediate species in the stepwise deoxygenation of Compound 3-1 to Compound 3-3. In terms of reaction rate, it was identified that deoxygenation from Compound 3-1 to Compound 3-2 occurred instantly at room temperature, and the conversion into Compound 3-3 took about 3 hours. According to previous studies of the inventors, the same reaction between CO (g) and Compound 3-1′ produces Compound 3-3′ within 1 hour, and therefore, it may be seen that the hardened ligand may affect an activation barrier for deoxygenation of the nickel(II) nitro species to the nickel nitrosyl compound.

Referring to FIG. 1 (c), the crystal structure of Compound 3-3 shows a 4-coordinate nickel-nitrosyl species, and mono-nitrosyl coordination was identified through nitrosyl vibration at 1,655 cm⁻¹. Compound 3-3 corresponds to a {Ni(NO)}¹⁰ complex according to the Enemark-Feltham notation since there are 10 electrons in the metal d and NO π* orbitals.

In order to explain the electronic structure of Compound 3-3, the crystal structure was analyzed. Noticeable distortion in the square planar shape for the nickel center (τ₀=0.58) (ref) and the half-bent bonding mode of NO (Ni1-N2-O1 bond angle=150.1(2)°) implies that Compound 3-3 may have a Ni(I) center.

FIG. 2 shows structural data for the nickel-nitrosyl moieties of Compound 3-3 and Compound 3-3′ according to the present disclosure and Ni K-edge X-ray absorption spectrum images of Compound 3-3 and Compound 3-3′ with respect to {(^{acri}PNP)Ni^{II}(CO)}{BF₄}, (^{acri}PNP)Ni^{I}(CO) and {^{acri}PNP)Ni⁰(CO)}{Na}.

Referring to FIG. 2 (b), the metric parameters (d_{N11-N2}2=1.683(2) Å and d_{N2-O1}=1.186(2) Å) indicate that Compound 3-3 has similar electronic properties to (PNP)Ni(NO) (Compound 3-3′, d_{Ni—N}=1.694(4) Å, d_{NO—O}=1.174(6) Å, ∠Ni—N—O=145.4(1)°) and (PNP′)Ni(NO) (PNP′=(^{t}Bu₂PCH₂SiMe₂)₂N⁻), d_{Ni—N}=1.692(4) Å, d_{NO—O}=1.185(5) Λ, ∠Ni—N—O=149.3(4)°), which are nickel(I)-nitroso species corresponding to a {Ni(NO)}¹⁰ complex.

Referring to FIGS. 2 (a) and (b), the angle of ∠Ni—NO measured in Compound 3-3 was identified to be larger compared to the angle of Compound 3-3′ (Ä (∠Ni—NO)=4.7°). Through this, it may be seen that Compound 3-3 has smaller electron density at the nitrogen atom of the nitrosyl moiety. When Ni—N≡O has cationic nitrosonium coordination, the corresponding angle becomes 180°. According to previous studies of the inventors on (PNP)Ni(NO), this is a considerably important factor in the conversion from imbalance of NO requiring direct attack of free nitric oxide on the nitrogen of the Ni—NO moiety, an important step for N₂O production.

Oxidation states of the nitrosyl species Compound 3-3 and Compound 3-3′ were further investigated by analyzing the pre-edge region through Ni K-edge X-ray absorption spectroscopy (XAS) (atom of Ni is electron is excited to Ni 3d orbital, about 8330 eV to 8335 eV). For accurate analysis, a series of nickel carbonyl species (^{acri}PNP)Niⁿ(CO) (n=0, +1 or +2) were used as standards for three different oxidation states of nickel ion supported by the ^{acri}PNP ligand. Through the pre-edge characteristics, it was identified that both Compound 3-3 and Compound 3-3′ have Ni(I) ions.

Referring to FIG. 2 (c), it may be seen that pre-edge energy appears at 8334.0 eV, which is almost similar to the energy of (^{acri}PNP)Ni^{I}(CO) at 8333.4 eV. On the other hand, other nickel carbonyl complexes do not show pre-edge characteristics. This comparison implies that the two nickel nitrosyl complexes are designated as Ni(I)—NO species.

As described above, it was identified that, using the (PNP)Ni catalyst, successful conversion for Ni—NO₃, Ni—NO₂ and Ni—NO, that is, all three Ni—NOₓ species of Compound 3-1 to Compound 3-3, was accomplished under mild conditions.

It was identified that NO transfer of Ni—NOₓ to the organic substrate using the (PNP)Ni catalyst was due to the open shell characteristics of Compound 3-3 limiting reactivity for CO (g). For conversion from NOₓ to NO, the entire reaction was performed under the CO atmosphere, and subsequently, a reaction of transferring the Ni-mediated nitroso group to an alkyl halide was performed as in the following Reaction Formula 2.

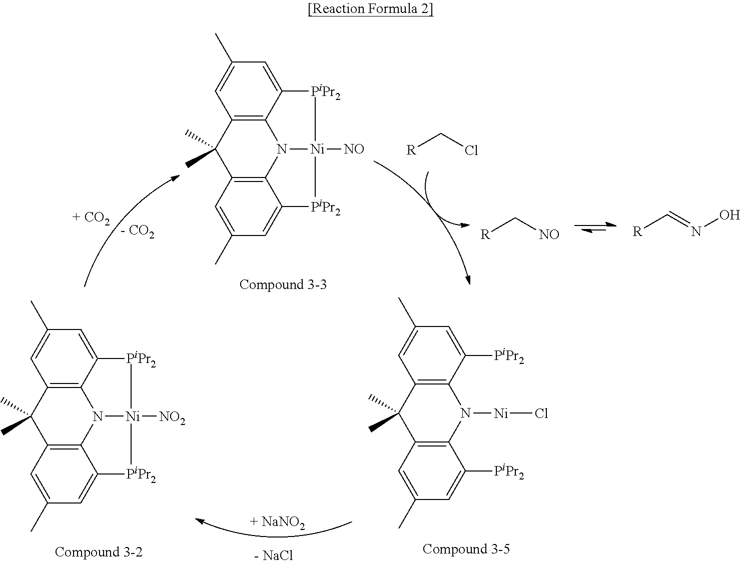

Referring to Reaction Formula 2, a radical type reaction was considered based on the fact that Compound 3-3 has Ni(I)—·NO characteristics. In order to identify this, Compound 3-3 and 10 equivalents of benzyl chloride were reacted in acetone-d₆, and the reaction was monitored for 48 hours using ³¹P NMR spectroscopy. Herein, the reaction temperature was adjusted to 60° C. Referring to Reaction Formula 1, Compound 3-5, that is, (^{acri}PNP)Ni(Cl), was slowly produced, reaching about 30% formation, and having a singlet at 40.5 ppm was identified. In addition, through ¹H NMR and GC-MS analyses of the reaction mixture, formation of benzaldehyde oxime as an organic product was identified. It may be seen that the NO group is transferred to the benzyl moiety, and then tautomerized to an oxime derivative, a product, as shown in Reaction Formula 2.

Referring to Reaction Formula 2, it was identified that Compound 3-5 produced in the NO transfer reaction may be converted into Compound 3-2 by the reaction with NaNO₂. In addition, since Compound 3-2 may be converted into Compound 3-3 through deoxygenation by CO (g), a catalytic cycle in which Ni—NOₓ is converted into Ni—NO, and the nitroso group is transferred to an alkyl halide was considered. Through this, it was identified that a technology of preparing an oxime derivative using nitrogen oxyanion as a NO supply source was able to be provided.

### EXAMPLE: PREPARATION OF OXIME DERIVATIVE/PREPARATION OF SECOND COMPOUND

### Example 1

1 mol % of Compound 3-5 was prepared as a Ni-based catalyst, 3 equivalents of sodium nitrite (NaNO₂) was prepared as a nitrogen oxide, and 3 mL of acetone or THF was prepared as a solvent. As a substrate, that is, a first compound, benzyl chloride was prepared. After that, Compound 3-5, the benzyl chloride and the sodium nitrite were added to the solvent, mixed, and reacted for 24 hours under a condition of 60° C. and 1 atmosphere of CO (g) to prepare an oxime derivative. Herein, the molar ratio between the catalyst, the substrate and the sodium nitrite were adjusted to 1:100:300.

### Example 2

An oxime derivative was prepared in the same manner as in Example 1, except that benzyl bromide was used as the substrate.

### Example 3

An oxime derivative was prepared in the same manner as in Example 1, except that (1-bromoethyl)benzene was used as the substrate.

### Example 4

An oxime derivative was prepared in the same manner as in Example 3, except that tetrahydrofuran (THF) was used as the solvent.

### Example 5

An oxime derivative was prepared in the same manner as in Example 3, except that propylene carbonate was used as the solvent.

### Example 6

An oxime derivative was prepared in the same manner as in Example 3, except that 1,4-dioxane was used as the solvent.

### Example 7

An oxime derivative was prepared in the same manner as in Example 3, except that toluene was used as the solvent.

### Example 8

An oxime derivative was prepared in the same manner as in Example 3, except that acetonitrile (MeCN) was used as the solvent.

### Example 9

An oxime derivative was prepared in the same manner as in Example 3, except that the reaction temperature was adjusted to 25° C.

### Example 10

An oxime derivative was prepared in the same manner as in Example 3, except that the reaction temperature was adjusted to 80° C.

### Example 11

An oxime derivative was prepared in the same manner as in Example 3, except that 2 equivalents of sodium nitrite was used as the nitrogen oxide.

### Example 12

An oxime derivative was prepared in the same manner as in Example 4, except that 12.5 mol % of 15-crown-5 was further added as a crown ether.

### Example 13

An oxime derivative was prepared in the same manner as in Example 4, except that 25 mol % of 15-crown-5 was further added as a crown ether.

### Example 14

An oxime derivative was prepared in the same manner as in Example 4, except that 50 mol %, of 15-crown-5 was further added as a crown ether.

### Example 15

An oxime derivative was prepared in the same manner as in Example 3, except that 0.5 mol % of Compound 3-5 was used as the Ni-based catalyst.

### Example 16

An oxime derivative was prepared in the same manner as in Example 3, except that 0.1 mol % of Compound 3-5 was used as the Ni-based catalyst.

### Example 17

An oxime derivative was prepared in the same manner as in Example 4, except that benzyl bromide was used as the substrate.

### Example 18

An oxime derivative was prepared in the same manner as in Example 4, except that 4-methylbenzyl bromide was used as the substrate.

### Example 19

An oxime derivative was prepared in the same manner as in Example 4, except that 4-fluorobenzyl bromide was used as the substrate.

### Example 20

An oxime derivative was prepared in the same manner as in Example 4, except that 4-(trifluoromethyl)benzyl bromide was used as the substrate.

### Example 21

An oxime derivative was prepared in the same manner as in Example 4, except that 4-(trifluoromethoxy)benzyl bromide was used as the substrate.

### Example 22

An oxime derivative was prepared in the same manner as in Example 4, except that methyl-4-(bromomethyl)benzoate was used as the substrate.

### Example 23

An oxime derivative was prepared in the same manner as in Example 4, except that diphenylbromomethane was used as the substrate.

### Example 24

An oxime derivative was prepared in the same manner as in Example 4, except that 9-bromofluorene was used as the substrate.

### Example 25

An oxime derivative was prepared in the same manner as in Example 4, except that iodocyclohexane was used as the substrate.

### Comparative Example 1

Preparation was made in the same manner as in Example 3, except that 4 equivalents of sodium nitrite was used as the nitrogen oxide.

### Comparative Example 2

Preparation was made in the same manner as in Example 3, except that the catalyst was not used.

### Comparative Example 3

Preparation was made in the same manner as in Example 4, except that α-nitrotoluene was used as the substrate.

### Experimental Example

### Mechanism Analysis

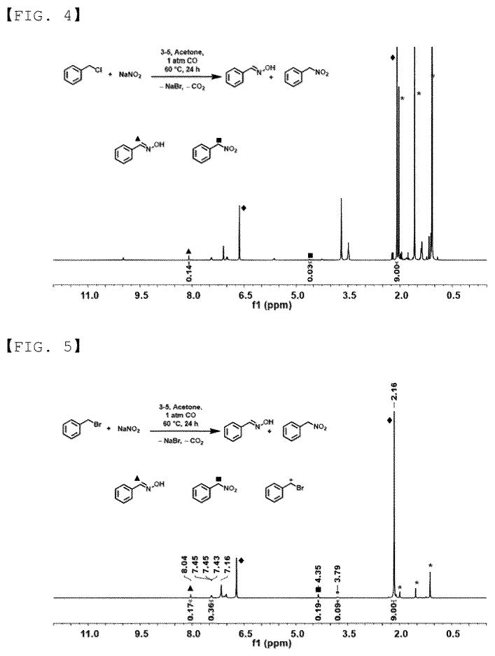

In order to more specifically understand the catalytic NO transfer from (^{acri}PNP)Ni(NO) (Compound 3-3) to an alkyl halide, experimental and theoretical evaluations were performed. As an initial step, two different reaction paths were assumed as shown in FIG. 3.

FIG. 3 schematically shows the two different reaction paths of the catalytic reaction according to the present disclosure. Specifically, FIG. 3 (a) shows a direct NC coupling path, FIG. 3 (b) shows a halide abstraction path, FIG. 3 (c) shows a plot of concentration versus time (thermal response) of Compound 3-3, and FIG. 4 (d) schematically shows electron density of Compound 3-3.

Referring to FIG. 3 (a), the nickel(I) center first abstracts halide from benzyl halide to release the NO radical, and then bonds to the benzyl radical. Nickel(I)-mediated C—C (and C—N) coupling is well known in organic-nickel cross-coupling reactions reported by several research groups.

Through the lack of reaction in several experiments using radical species such as TEMPO catalyst (2,2,6,6-tetramethylpiperidin-1-oxyl, Co₉H₁₈NO) and 2,4,6-tri-tert-butylphenoxyl radicals, it may be seen that strong antiferromagnetic coupling between Ni(I) and ·NO in Compound 3-3 may significantly reduce reactivity.

Referring to FIG. 3 (b), as another path, a case of the nitroso group of Compound 3-3 directly attacking the benzyl position of benzyl chloride to form a nitrosylated organic product may be considered. To obtain information on dynamics, a reaction between 10 equivalents of benzyl bromide and Compound 3-3 was performed in acetone-d₆. Herein, the reaction temperature was adjusted to 60° C. The reaction was monitored by ³¹P NMR spectroscopy, and a pseudo first order decay was obtained for the consumption of Compound 3-3 with a reaction constant of 1×10⁻⁵ s⁻¹. However, only 25% conversion of Compound 3-3 was observed after 6 hours, which implies that this step is a rate-determining step. When the reaction follows this path, a nickel C-nitroso species (Ni—N(O)R) will be produced rarely according to the literature, and actually, (^{acri}PNP)Ni(NO^{t}Bu) was synthetically prepared from the reaction between (^{acri}PNP)Ni and NO^{t}Bu. According to the XRD data, there is a square planar nickel center having N-bonding tert-nitrosobutane with a Ni—N bond length of 1.890(4) Å.

Referring to FIG. 3 (d), it may be seen that SOMO (Singly Occupied Molecular Orbital) has spin density localized mostly in the NO moiety. This implies that coordination of tert-nitrosobutane to the nickel(I) center provides a Ni(II)—·NO species. Since a nickel nitrosoalkane species is reasonably separable, it may be seen that the direct N—C coupling is a reasonable path.

As described above, it was identified that NO₃⁻ was able to be directly converted into NO through deoxygenation using CO (g) generated from a single nickel ion. In other words, it was identified that sequential conversion of the nickel nitrate species (^{acri}PNP)Ni(ONO₂) (Compound 3-1) into the nitro species (Compound 3-2), and ultimately into the nitrosyl species (^{acri}PNP)Ni(NO) (Compound 3-3) occurred through the stepwise movement of oxygen atom using CO (g). In addition, it was identified that Compound 3-3 was able to nitrosylate an alkyl halide to form an oxime product. Furthermore, it was identified that nickel(II) halide (^{acri}PNP)Ni(Cl) (Compound 3-5) may be used as an efficient catalyst for catalytically forming a new C—N bond by using NO₂ as a nitroso source and converting an alkyl halide to an oxime. Through this, it may be seen that the method for preparing an oxime derivative according to the present disclosure is capable of providing a new method of utilizing NOₓ in nickel-mediated catalysis to produce useful value-added organic products.

### Structural Analysis and TON Calculation

For the products according to Example 1 to Example 5, Example 8 to Example 14, Example 16 to Example 25 and Comparative Example 1 to Comparative Example 3, structures of the products were analyzed through ¹H NMR analysis using mesitylene as an internal standard and GC analysis, and TONs (turnover number, number of moles of oxime formed per mole of Ni catalyst) were calculated.

FIG. 4 is a ¹H NMR analysis image for the product according to Example 1.

2.6981 g of a sample obtained from 8.1021 g of the crude product solution according to Example 1 was concentrated under vacuum, then the sample was dissolved in C₆D₆, and 0.143 mmol of mesitylene was added as an internal standard for ¹H NMR analysis.

Referring to FIG. 4, formation of benzaldehyde oxime (▴) and α-nitrotoluene (▪) was identified through the results obtained after catalytic NO transfer from benzyl chloride (●). Herein, ‘*’ is diacetone alcohol formed through aldol condensation of acetone.

The NMR ratio of oxime:mesityiene=0.14:1 indicates that 0.020 mmol of oxime is included in 2.6981 g of the sample. Through this, it was calculated that the total amount of oxime present in 8.1021 g of the crude product solution was 0.060 mmol, and the TON (mol of oxime/5.33 μmol of Compound 3-5) was 11, and the results are shown in Table 1.

After that, for the products according to Example 2 to Example 5, Example 8 to Example 14, Example 16 to Example 25 and Comparative Example 1 to Comparative Example 3, the structures of the products were analyzed by performing ¹H NMR analysis and GC analysis in the same manner as the analysis method for the product according to Example 1, except that the total amounts of the crude product solution and the sample and the number of moles of the internal standard were varied, and TONs were calculated. The results are shown in Table 1. Herein, acetone-d₆ was used in Example 24 and DMSO-d₆ was used in Example 25 when performing the ¹H NMR analysis.

TABLE 1

| | Product | | Sample | | | | TON |
|---|---|---|---|---|---|---|---|
| | Total | | Total | | ¹H NMR | | (GC |
| | Amount | Oxime | Amount | Oxime | Mesitylene | | yield |
| | (g) | (mmol) | (g) | (mmol) | (mmol) | Oxime:Mesitylene | (%)) |
| Example 1 | 8.1021 | 0.060 | 2.6981 | 0.020 | 0.143 | 0.14:1 | 11 |
| Example 2 | 6.261 | 0.270 | 0.814 | 0.035 | 0.208 | 0.17:1 | 51 |
| Example 3 | 10.3503 | 0.40 | 1.5215 | 0.0583 | 0.108 | 0.54:1 | 75 |
| Example 4 | 6.270 | 0.079 | 2.7302 | 0.0343 | 0.143 | 0.24:1 | 15 |
| Example 5 | 10.2715 | 0.315 | 3.4578 | 0.106 | 0.143 | 0.74:1 | 59(54) |
| Example 8 | 4.6398 | 0.363 | 1.8276 | 0.143 | 0.143 | 1:1 | 68 |
| Example 9 | 6.3156 | 0.055 | 2.4695 | 0.0214 | 0.143 | 0.15:1 | 10 |
| Example 10 | 4.9284 | 0.289 | 1.9762 | 0.116 | 0.143 | 0.81:1 | 54(44) |
| Example 11 | 6.9368 | 0.213 | 2.3310 | 0.0715 | 0.143 | 0.50:1 | 40 |
| Example 12 | 9.8377 | 0.039 | 5.0398 | 0.020 | 0.143 | 0.14:1 | 7 |
| Example 13 | 7.9854 | 0.049 | 3.2335 | 0.020 | 0.143 | 0.14:1 | 9 |
| Example 14 | 7.5486 | 0.171 | 1.7681 | 0.040 | 0.143 | 0.28:1 | 32 |
| Example 16 | 5.663 | 0.125 | 1.995 | 0.044 | 0.143 | 0.31:1 | 234 |
| Example 17 | 3.984 | 0.123 | 0.842 | 0.026 | 0.143 | 0.18:1 | 23 |
| Example 18 | 5.886 | 0.100 | 1.115 | 0.019 | 0.082 | 0.23:1 | 19 |
| Example 19 | 4.914 | 0.100 | 1.142 | 0.023 | 0.065 | 0.36:1 | 19 |
| Example 20 | 7.620 | 0.176 | 1.863 | 0.043 | 0.080 | 0.54:1 | 33 |
| Example 21 | 5.977 | 0.135 | 1.954 | 0.044 | 0.143 | 0.31:1 | 25 |
| Example 22 | 5.089 | 0.076 | 0.940 | 0.014 | 0.143 | 0.1:1 | 14 |
| Example 23 | 13.70 | 0.234 | 3.014 | 0.051 | 0.143 | 0.36:1 | 44 |
| Example 24 | 5.92 | 0.424 | 1.857 | 0.133 | 0.133 | 1:1 | 80 |
| Example 25 | 6.207 | 0.324 | 1.424 | 0.13 | 0.143 | 0.52:1 | 61 |
| Comparative | — | — | — | — | — | — | — |
| Example 1 | | | | | | | |
| Comparative | — | — | — | — | — | — | — |
| Example 2 | | | | | | | |
| Comparative | — | — | — | — | — | — | — |
| Example 3 | | | | | | | |

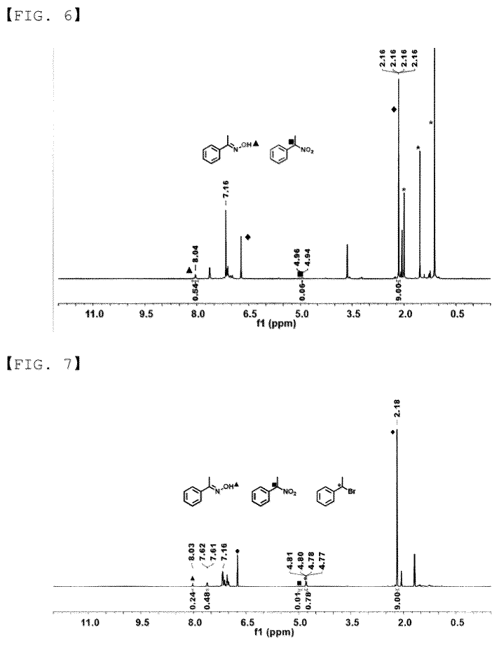

FIG. 5 is a H NMR analysis image for the product according to Example 2.

Referring to FIG. 5, formation of benzaldehyde oxime (▴) and α-nitrotoluene (▪) was identified through the results obtained after catalytic NO transfer from benzyl bromide (●). Herein, ‘*’ is diacetone alcohol formed through aldol condensation of acetone.

FIG. 5 is a ¹H NMR analysis image for the product according to Example 3.

Referring to FIG. 6, formation of acetophenone oxime (▴) and (1-nitroethyl)benzene (▪) was identified through the results obtained after catalytic NO transfer. Herein, ‘*’ is diacetone alcohol formed through aldol condensation of acetone, and ‘♦’ is mesitylene.

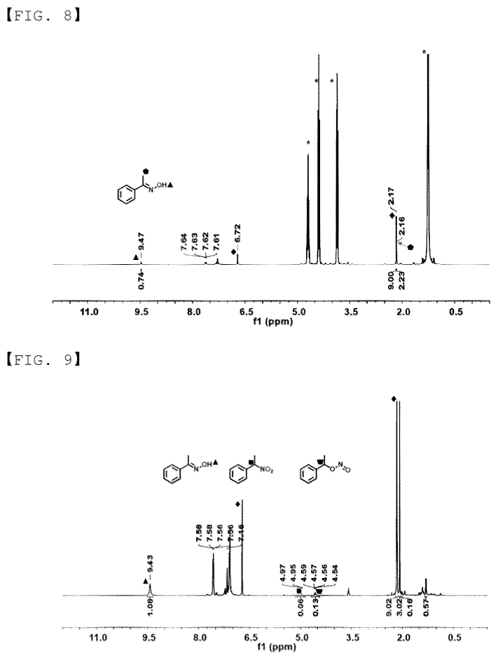

FIG. 7 is a ¹H NMR analysis image for the product according to Example 4.

Referring to FIG. 7, formation of benzaldehyde oxime (▴) and (1-nitroethyl)benzene (▪) was identified through the results obtained after catalytic NO transfer. Herein, ‘♦’ is mesitylene.

FIG. 8 is a ¹H NMR analysis image for the product according to Example 5.

Referring to FIG. 8, formation of acetophenone oxime (▴) was identified through the results obtained after catalytic NO transfer. Herein, ‘*’ is propylene carbonate, and ‘♦’ is mesitylene.

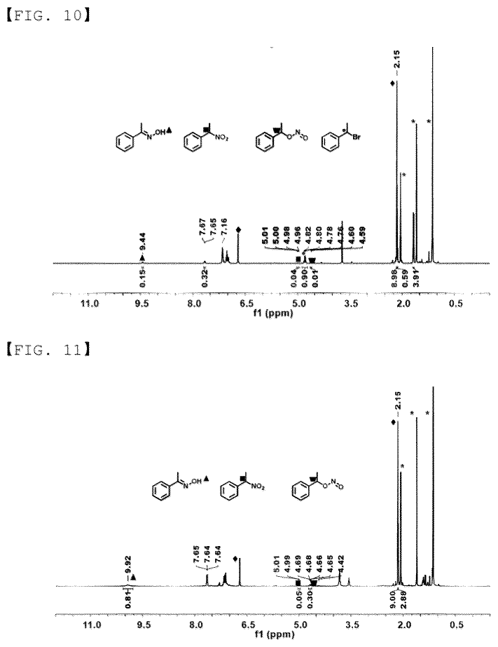

FIG. 9 is a ¹H NMR analysis image for the product according to Example 8.

Referring to FIG. 9, formation of acetophenone oxime (▴) was identified through the results obtained after catalytic NO transfer. Herein, ‘*’ is diacetone alcohol formed through aldol condensation of acetone, and ‘♦’ is mesitylene.

FIG. 10 is a ¹H NMR analysis image for the product according to Example 9.

Referring to FIG. 10, formation of acetophenone oxime (▴) was identified through the results obtained after catalytic NO transfer. Herein, ‘*’ is diacetone alcohol formed through aldol condensation of acetone, and ‘♦’ is mesitylene.

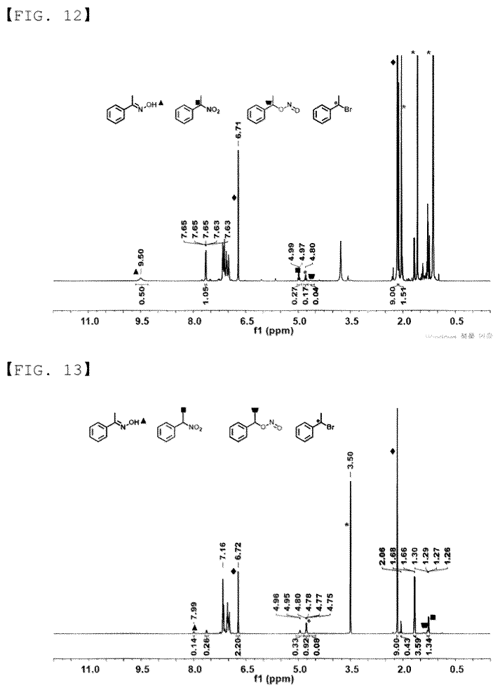

FIG. 11 is a ¹H NMR analysis image for the product according to Example 10.

Referring to FIG. 11, formation of acetophenone oxime (▴) was identified through the results obtained after catalytic NO transfer. Herein, ‘*’ is diacetone alcohol formed through aldol condensation of acetone, and ‘♦’ is mesitylene.

FIG. 12 is a ¹H NMR analysis image for the product according to Example 11.

Referring to FIG. 12, formation of acetophenone oxime (▴) was identified through the results obtained after catalytic NO transfer. Herein, ‘*’ is diacetone alcohol formed through aldol condensation of acetone, and ‘♦’ is mesitylene.

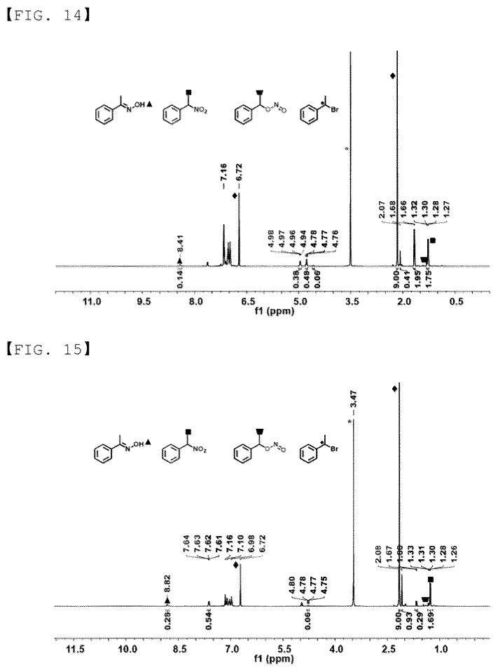

FIG. 13 is a ¹H NMR analysis image for the product according to Example 12.

Referring to FIG. 13, formation of acetophenone oxime (▴) was identified through the results obtained after catalytic NO transfer. Herein, ‘*’ is crown ether, and ‘♦’ is mesitylene.

FIG. 14 is ¹H NMR analysis image for the product according to Example 13.

Referring to FIG. 14, formation of acetophenone oxime (▴) was identified through the results obtained after catalytic NO transfer. Herein, ‘*’ is crown ether, and ‘♦’ is mesitylene.

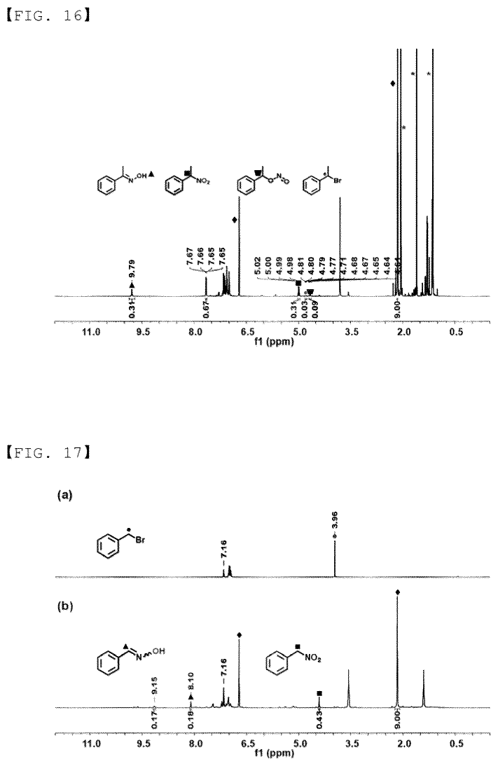

FIG. 15 is a ¹H NMR analysis image for the product according to Example 14.

Referring to FIG. 15, formation of acetophenone oxime (▴) was identified through the results obtained after catalytic NO transfer. Herein, ‘*’ is crown ether, and ‘♦’ is mesitylene.

FIG. 16 is ¹H NMR analysis image for the product according to Example 16.

Referring to FIG. 16, formation of acetophenone oxime (▴) was identified through the results obtained after catalytic NO transfer. Herein, ‘*’ is diacetone alcohol formed through aldol condensation of acetone, and ‘♦’ is mesitylene.

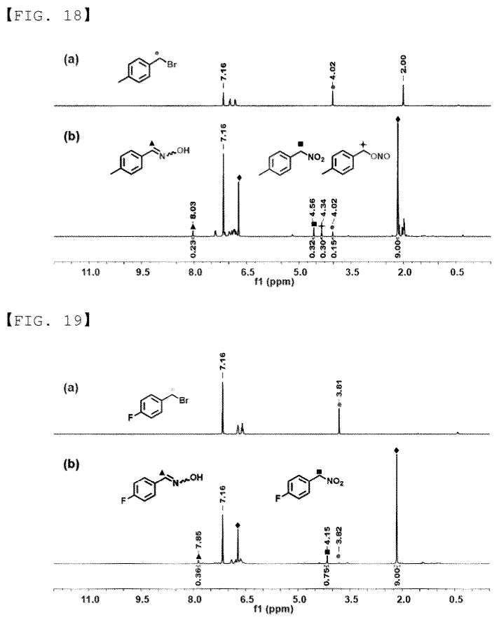

FIG. 17 is ¹H NMR analysis image for the product according to Example 17.

Referring to FIG. 17, formation of benzaldehyde oxime (▴) and α-nitrotoluene (▪) was identified through the results obtained after catalytic NO transfer from benzyl bromide (●). Herein, ‘♦’ is mesitylene.

FIG. 18 is a ¹H NMR analysis image for the product according to Example 18.

Referring to FIG. 18, formation of 4-methylbenzaldehyde oxime (▴), 1-methyl-4-(nitromethyl)benzene (▪) and 4-methylbenzyl nitrite ( [symbol] ) was identified through the results obtained after catalytic NO transfer from 4-methylbenzyl bromide (●). Herein, ‘♦’ is mesitylene.

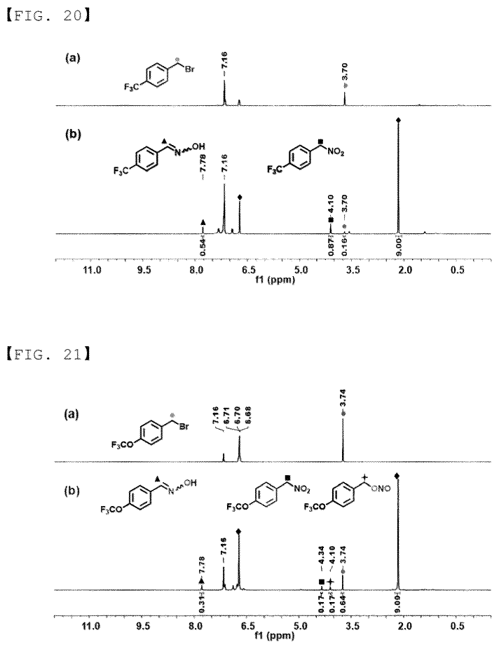

FIG. 19 is a ¹H NMR analysis image for the product according to Example 19.

Referring to FIG. 19, formation of 4-fluorobenzaldehyde oxime (▴) and 1-fluoro-4-(nitromethyl)benzene (▪) was identified through the results obtained after catalytic NO transfer from 4-fluorobenzyl bromide (●). Herein, ‘♦’ is mesitylene.

FIG. 20 is a ¹H NMR analysis image for the product according to Example 20.

Referring to FIG. 20, formation of 4-trifluoromethylbenzaldehyde oxime (▴) and 1-trifluoromethyl-4-(nitromethyl)benzene (▪) was identified through the results obtained after catalytic NO transfer from 4-(trifluoromethyl)benzyl bromide (●). Herein, ‘♦’ is mesitylene.

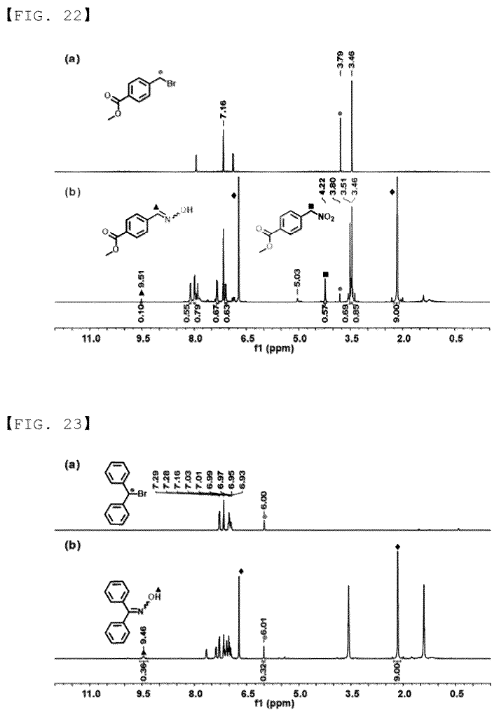

FIG. 21 is a ¹H NMR analysis image for the product according to Example 21.

Referring to FIG. 21, formation of 4-trifluoromethoxybenzaldehyde oxime (▴), 1-trifluoromethoxy-4-(nitromethyl)benzene (▪) and 4-trifluoromethoxybenzyl nitrite ( [symbol] ) was identified through the results obtained after catalytic NO transfer from 4-(trifluoromethoxy)benzyl bromide (●). Herein, ‘♦’ is mesitylene.

FIG. 22 is a ¹H NMR analysis image for the product according to Example 22.

Referring to FIG. 22, formation of methyl 4-((hydroxyimino)methyl)benzoate (▴) and methyl 4-(nitromethyl)benzoate (▪) was identified through the results obtained after catalytic NO transfer from methyl-4-(bromomethyl)benzoate (●). Herein, ‘♦’ is mesitylene.

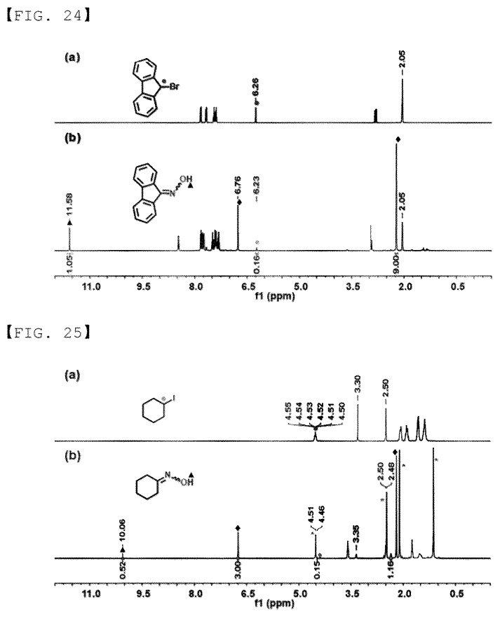

FIG. 23 is a ¹H NMR analysis image for the product according to Example 23.

Referring to FIG. 23, formation of benzophenone oxime (▴) was identified through the results obtained after catalytic NO transfer from diphenylbromomethane (●). Herein, ‘♦’ is mesitylene.

FIG. 24 is a ¹H NMR analysis image for the product according to Example 24.

Referring to FIG. 24, formation of 9-fluorenone oxime (▴) was identified through the results obtained after catalytic NO transfer from 9-bromofullerene (●). Herein, ‘♦’ is mesitylene.

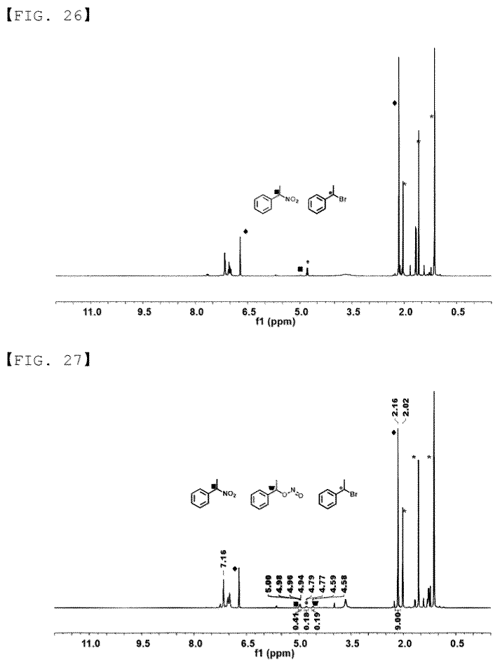

FIG. 25 is a ¹H NMR analysis image for the product according to Example 25.

Referring to FIG. 25, formation of 9-fluorenone oxime (▴) was identified through the results obtained after catalytic NO transfer from iodocyclohexane (●). Herein, ‘*’ is diacetone alcohol formed through aldol condensation of acetone, and ‘♦’ is mesitylene.

FIG. 26 is a ¹H NMR analysis image for the product according to Comparative Example 1.

Referring to FIG. 26, formation of (1-nitroethyl)benzene (▪) was identified through the results obtained after catalytic NO transfer from (1-bromoethyl)benzene (●), however, it was impossible to measure an oxime.

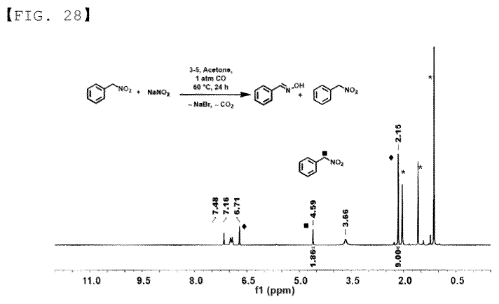

FIG. 27 is a ¹H NMR analysis image for the product according to Comparative Example 2.

Referring to FIG. 27, formation of (w-nitroethyl)benzene (▪) was identified through the results obtained after NO transfer from (1-bromoethyl)benzene (●), however, it was impossible to measure an oxime.

FIG. 28 is a ¹H NMR analysis image for the product according to Comparative Example 3.

Referring to FIG. 28, it was impossible to measure an oxime through the results obtained after catalytic NO transfer from α-nitrotoluene (▪).

### Yield, Selectivity and Turnover Number Analyses

For Examples 1 to 25 and Comparative Examples 1 to 0.3, yield of oxime and RNO₂, oxime selectivity, and TON (turnover number, number of moles of oxime formed per mole of Ni catalyst) were analyzed through ¹H NMR spectroscopy using mesitylene as an internal standard and GC analysis, and the results are shown in Table 2. Herein, when the measurement was not possible due to an extremely small amount, it was indicated as “-”.

TABLE 2

| | Yield (%) | | Oxime | TON |
|---|---|---|---|---|
| | Oxime | Nitroalkane | Selectivity (%) | (%) |
| Example 1 | 11 | 1 | 90 | 11 |
| Example 2 | 51 | 28 | 65 | 51 |
| Example 3 | 75 | 8 | 90 | 75 |
| Example 4 | 15 | 1 | 94 | 15 |
| Example 5 | 54 | — | — | 54 |
| Example 6 | 11 | 13 | 46 | 13 |
| Example 7 | 8 | 8 | 50 | 8 |
| Example 8 | 68 | 9 | 88 | 68 |
| Example 9 | 10 | 3 | 77 | 10 |
| Example 10 | 44 | 13 | 77 | 44 |
| Example 11 | 40 | 25 | 62 | 40 |
| Example 12 | 7 | 23 | 23 | 7 |
| Example 13 | 9 | 39 | 19 | 9 |
| Example 14 | 32 | 64 | 33 | 32 |
| Example 15 | 55 | 11 | 83 | 110 |
| Example 16 | 23 | 31 | 43 | 234 |
| Example 17 | 23 | 28 | 46 | 23 |
| Example 18 | 19 | 25 | 43 | 19 |
| Example 19 | 19 | 20 | 49 | 19 |
| Example 20 | 33 | 27 | 55 | 33 |
| Example 21 | 25 | 25 | 50 | 25 |
| Example 22 | 14 | 42 | 27 | 14 |
| Example 23 | 44 | — | >99 | 44 |
| Example 24 | 80 | — | >99 | 80 |
| Example 25 | 61 | 6 | 95 | 61 |
| Comparative | — | — | — | — |
| Example 1 | | | | |
| Comparative | — | 40 | — | — |
| Example 2 | | | | |
| Comparative | — | 78 | — | — |
| Example 3 | | | | |

Referring to Table 2, it was identified that, in Example 1, benzyl chloride was converted to benzaldehyde oxime with a yield of 11% and selectivity of 90%. When using benzyl chloride as a substrate, the TON for oxime formation was 11.

In Example 2, it was identified that selectivity of 64% and a higher TON of 51 were accomplished when using benzyl bromide as a substrate. Through this, it may be seen that a better leaving group is advantageous.

Meanwhile, it was identified that S_{N}2 type substitution of the halide group by nitrite anion causes a side reaction forming α-nitrotoluene as a by-product. Using benzyl bromide as a substrate produced more by-product compared to the reaction with benzyl chloride, and this was identified to be related to a leaving group effect. In addition, it was identified that (1-bromoethyl)benzene had a TON of 75 in the catalytic reaction with Compound 3-5, showing a higher catalytic conversion rate into acetophenone oxime.

In Example 3, the sterically hindered benzyl position significantly suppressed the side reaction to 8% as expected, and it was identified that selectivity for oxime formation was maintained at 90%. Accordingly, (1-bromoethyl)benzene was selected as a substrate for further studies.

In Example 4 to Example 8, various solvents were used to optimize the method for preparing an oxime derivative. Through this, it was identified that acetone and THF were particularly suitable.

In order to identify the effect of temperature changes on the oxime derivative preparation, the reaction temperature was adjusted. Lowering the reaction temperature was expected to reduce the formation of nitroalkane. In Example 9, it was identified that the reaction at room temperature proceeded slowly with oxime formation of 10%, however, the side reaction remained the same. In Example 10, interestingly, it was identified that production of both oxime and nitroalkane decreased when increasing the reaction temperature to 80° C. It may be seen that this is due to another side reaction including acetone and sodium nitrite as a base of the reaction medium. In other words, it was identified that aldol condensation of base-induced acetone produced diacetone alcohol, a main product, when using acetone as a solvent.

The content of sodium nitrite as nitrogen oxyanion was adjusted to obtain higher selectivity and TON. In Example 12 using 2 equivalents of sodium nitrite, obtaining an oxime yield of 40% and a TON of 40 was identified, however, in Comparative Example 1 using 4 equivalents of sodium nitrite, it was identified that only a trace amount of oxime formation was detected.

Meanwhile, in Example 12 to Example 14 using THE as a solvent to avoid the reaction between nitrite anion and acetone, the content of 15-crown-5 as an additive was adjusted to 1.2.5 mol %, 25 mol % and 50 mol %, respectively. It was identified that Example 14 having the 15-crown-5 content of 50 mol % had increased production of both oxime and nitroalkane compared to Example 12 having the 15-crown-5 content of 12.5 mol %, and Example 13 having the 15-crown-5 content of 25 mol %.

In order to examine the effect of catalyst amount on the oxime derivative preparation, in Example 15 and Example 16 using 0.5 mol % and 0.1 mol % of Compound 3-5 as the catalyst, it was identified that 234, the best TON, was obtained as the reaction time increased.

In order to identify the substrate range and limitation of the catalyst system, oxime derivatives were prepared using various substrates. In Example 17 using THE as a solvent, it was identified that benzaldehyde oxime was formed from unsubstituted benzyl bromide in a yield of 23% and selectivity of 46%. It was identified that having small steric hindrance around the benzyl position is advantageous for both the catalytic NO transfer and the side reaction of producing nitroalkane caused by direct substitution with nitrite anion. In Example 18 to Example 21 using a substrate substituted with a different group at the para position of benzyl bromide, it was identified that a lower TON was generally observed. Similarly, in Example 22 using methyl 4-(bromomethyl)benzoate as a substrate, it was identified that an oxime was produced with relatively low selectivity and a TON of 14 was observed.

In contrast, in Example 23 and Example 24 using diphenylbromomethane and 9-bromofullerene as a substrate, it was identified that selectivity for NO-transfer dramatically increased when steric hindrance increased at the benzyl position. Particularly, in Example 23, it was identified that only the oxime product had a yield of 44% and selectivity of greater than 99%. Similarly, in Example 24 using 9-bromofluorene as a substrate, it was identified that a result of exclusively forming fluorenone oxime having a higher TON of 80 with a yield of 44% and selectivity of greater than 99% was observed. Meanwhile, in Example 25 using iodocyclohexane, a substrate with no benzyl position, it was identified that cyclohexanone oxime was selectively produced with a yield of about 61% at a slightly higher reaction temperature of 80° C.

In contrast, in Comparative Example 1 in which a Ni-based catalyst was not used, it was identified that oxime formation was not observed. Through this, it may be seen that the reaction of preparing an oxime derivative according to the present disclosure is accelerated by a Ni-based catalyst. In addition, in Comparative Example 3 using α-nitrotoluene instead of benzyl bromide as a substrate, it was identified that oxime formation was not observed.

In other words, through the method for preparing an oxime derivative according to the present disclosure, it may be seen that a successful catalytic NO-transfer reaction as the following Reaction Formula 3 to form value-added organic products from a nickel-nitrosyl complex is possible.

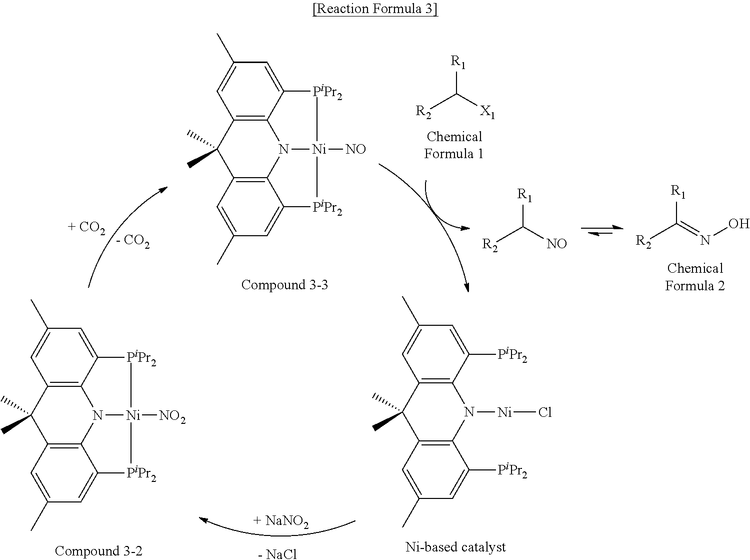

The detailed description provided above illustrates and explains the present disclosure. In addition, the above-provided description merely shows and explains preferred embodiments of the present disclosure, and the present disclosure may be used under various other combinations, changes and environments as described above, and changes or modifications may be made within the scope of the inventive concept disclosed in the present specification, the scope equivalent to the disclosure described above and/or the scope of skill or knowledge in the art. Accordingly, the detailed description of the invention provided above is not intended to Limit the present disclosure to the disclosed embodiments. In addition, accompanying claims need to be construed to include other embodiments as well.

1. A method for preparing an oxime derivative, the method comprising: preparing a second compound represented by the following Chemical Formula 2 from a first compound represented by the following Chemical Formula 1,
wherein the preparing of a second compound uses a Ni-based catalyst represented by the following Chemical Formula 3: 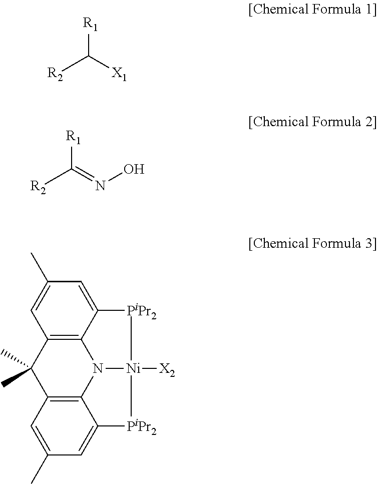
in Chemical Formula 1 to Chemical Formula 3,
R₁ and R₂ are each independently hydrogen; an alkyl group having 1 to 6 carbon atoms; an alkyl group having 1 to 3 carbon atoms substituted with an unsubstituted or substituted aryl group having 6 to 30 carbon atoms; or an unsubstituted or substituted aryl group having 6 to 30 carbon atoms; or
R₁ and R₂ are linked to each other to form an alicyclic ring having 5 to 12 carbon atoms; or an alicyclic ring having 5 to 12 carbon atoms to which an aromatic ring having 6 to 30 carbon atoms bonds; and
X₁ and X₂ are each independently F; Cl; Br; or I;
in Chemical Formula 3, iPr represents an iso-propyl group or related substituent; and
in the substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ is an alkyl group having 1 to 3 carbon atoms.

2. The method of claim 1, wherein, in Chemical Formula 1,
X₁ is Br;
R₁ and R₂ are each independently hydrogen; or an unsubstituted or substituted aryl group having 6 to 30 carbon atoms; and
in the substituted aryl group having 6 to 30 carbon atoms, the substituent is F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or CO₂Rₐ, and Rₐ is an alkyl group having 1 to 3 carbon atoms.

3. The method of claim 2, wherein, in Chemical Formula 1,
at least one of R₁ and R₂ is 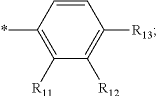
R₁₁ to R₁₃ are each independently hydrogen; F; Cl; Br; I; an alkyl group having 1 to 3 carbon atoms unsubstituted or substituted with at least one of F, Cl, Br and I; an alkoxy group having 1 to 3 carbon atoms unsubstituted or substituted with at least one of F, Cl, Br and I; or —CO₂Rₐ, and Rₐ is an alkyl group having 1 to 3 carbon atoms; and
“*” represents a bonding position.

4. The method of claim 1, wherein, in Chemical Formula 1,
X₁ is Br; and
R₁ and R₂ are linked to each other to form an alicyclic ring having 5 to 12 carbon atoms; or an alicyclic ring having 5 to 12 carbon atoms to which an aromatic ring having 6 to 30 carbon atoms bonds.

5. The method of claim 1, wherein R₁ and R₂ are linked to each other to form 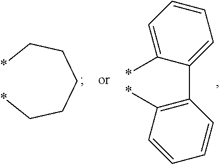 [SMILES: *CCCCC* ; *c1ccccc1-c1ccccc1*]
and “*” represents a bonding position.

6. The method of claim 1, wherein the first compound is one selected from among compounds represented by the following Chemical Formula 1-1 to Chemical Formula 1-10: 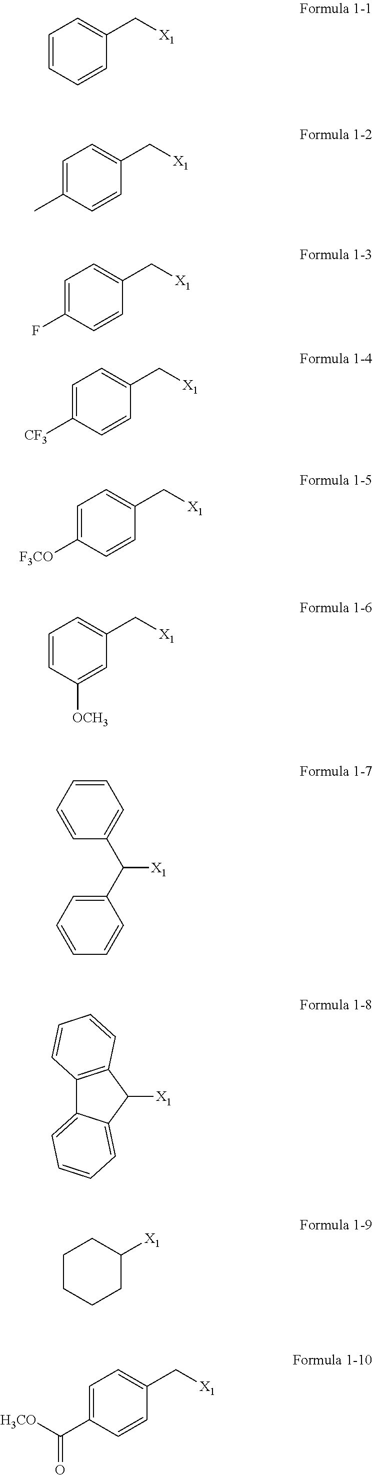
in the chemical formulae,
X₁ is F; Cl; Br; or I.

7. The method of claim 1, wherein the first compound is one selected from among the following Compound 1-1 to Compound 1-10:

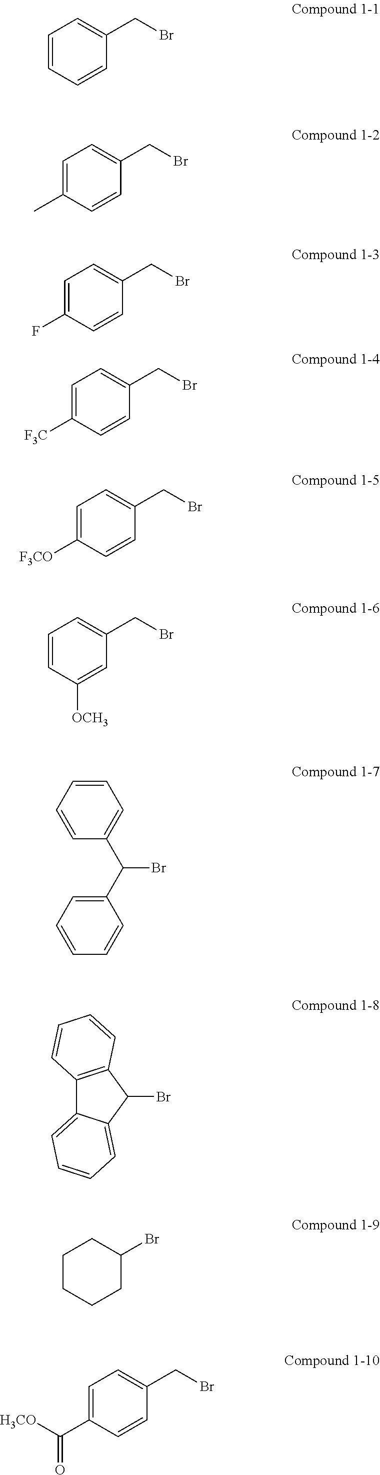

8. The method of claim 1, wherein the second compound is one selected from among the following Compound 2-1 to Compound 2-10:

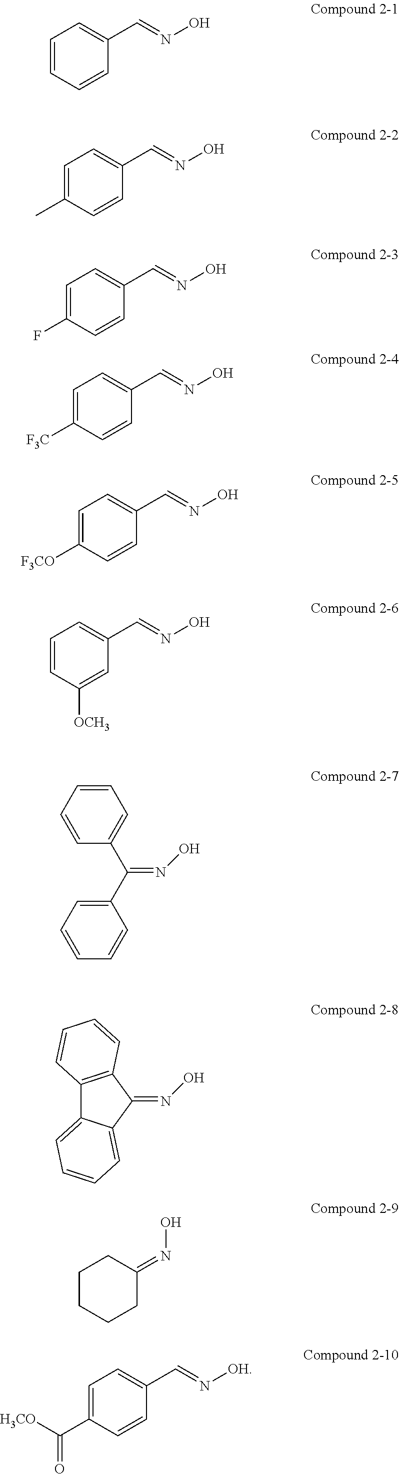

9. The method of claim 1, wherein the preparing of a second compound forms the second compound from the first compound using a mixture solution including the Ni-based catalyst, a nitrogen oxide and a solvent.

10. The method of claim 9, wherein the first compound and the nitrogen oxide have a molar ratio of greater than or equal to 1:2 and less than 1:4.

11. The method of claim 9, wherein the solvent includes at least one of acetone, tetrahydrofuran, propylene carbonate, 1,4-dioxane, toluene and acetonitrile.

12. The method of claim 9, wherein the nitrogen oxide is nitrogen oxyanion, the solvent is tetrahydrofuran, and the mixture solution further includes a crown ether.

13. The method of claim 1, wherein the preparing of a second compound is performed for a period of 24 hours or more and 48 hours or less at a temperature of 25° C. or higher and 80° C. or lower under a CO atmosphere.
